(19) United States
(12) Patent Application Publication
(10) Pub. No.: US 2023/0104323 A1
(43) Pub. Date: Apr. 6, 2023

(54) FORMULATIONS OF POLYBASIC DRUGS TO REDUCE MULTI-ORGAN TOXICITY

(71) Applicant: MANU CHAUDHARY, Panchkula (IN)

(72) Inventor: MANU CHAUDHARY, Panchkula (IN)

(21) Appl. No.: 17/785,100

(22) PCT Filed: Dec. 13, 2020
(86) PCT No.: PCT/IN2020/051027

§ 371 Date: Jun. 14, 2022

(30) Foreign Application Priority Data
Dec. 14, 2019 (IN) 201911051914

(51) Int. Cl. A61K 38/12; A61K 31/198; A61K 31/4172; A61K 47/36; A61K 31/7036; A61K 31/7048; A61P 39/00

(52) U.S. Cl. CPC A61K 38/12; A61K 31/198; A61K 31/4172; A61K 47/36; A61K 31/7036; A61K 31/7048; A61P 39/00

(57) ABSTRACT

The present invention relates to compositions and formulations of polybasic drugs to reduce multiorgan toxicity by making supramolecular cationic complex without covalent bond formation, without conjugation and without chemical modification of macromolecular entity used. The compositions and formulations made thereof act by multiple mechanisms simultaneously to reduce toxicity of cationic antibiotic drugs.

### FIELD OF THE INVENTION

The present invention relates to compositions and formulations made thereof to reduce multi organ toxicity caused by polybasic antibiotic drugs achieved by supramolecular cationic complex. The compounds of such compositions and formulation made thereof simultaneously act by multiple mechanisms to establish homeostasis in multiple organs to prevent toxicity. Particularly, the invention relates to formulation optimization and method of delivery of polybasic drugs to prevent nephro-, neuro- and oto-toxicity.

### BACKGROUND OF THE INVENTION

Aminoglycosides and polymyxins, commonly known as polybasic drugs, are broad-spectrum antibiotics commonly used for the treatment of serious bacterial infections. Failure of kidneys to eliminate aminoglycosides and polymyxins result in high blood levels or in higher intracellular drug concentrations in the proximal tubule due to drug accumulation and/or in other parts of the nephron, even with therapeutic doses, that can cause further renal and vestibular damage. In order to circumvent the problems associated with multi-organ toxicity, extensive research was performed and is still underway to improve structure of compounds to overcome toxicity.

In patent WO 2013/191550 A1, a novel one-step regio-selective chemical diazotation of the C3-amine functionality of the 2-DOS ring is presented to modify aminoglycosides. In patent US 2013/03455411 A1, a multi-step strategy involving the use of basic carbohydrate building blocks and a glycosylation reaction between the 1″-position of ring III and the 06 of ring II to develop novel aminoglycoside arbekacin.

In patent WO 2011/143497 A1, 140 mono- and difunctionalized derivatives of gentamycin were synthesized by modifying its N1- and/or N6′-positions.

To expand the scope of structural modifications of parent aminoglycoside, in patent WO 2014/013495 A1, work was done to synthesize various cationic amphiphilic derivatives of various aminoglycosides drugs without the use of an extensive number of synthetic steps to target the negatively-charged lipopolysaccharides present in bacterial cell wall through ionic interactions.

In patent WO 2011/044501 A2, in order to accelerate the process of developing new antibiotics, a chemical strategy was developed for synthesizing various neomycin analogues.

Later on, derivatives of patent WO 2011/044501 A2 novel plazomicin was synthesized by Achaogen. The pharmacokinetic evaluation and safety monitoring of plazomicin injection in healthy subjects showed no nephrotoxicity and ototoxicity in humans.

In patent WO 2014/1454713 A2, a novel chemical synthetic approach was used to develop analogues of sisomycin for alleviating aminoglycoside ototoxicity while preserving antibacterial activity. Recent evidence implies mitochondrial protein synthesis as a key element in aminoglycoside ototoxicity, and the defective mitochondrial function leads to reactive oxygen species (ROS) generation resulting in ototoxicity.

In patent WO 2013/170985 A1, the activities and ototoxicity of various drugs especially apramycin is disclosed. In patent WO 2011/124986 A2 addition to the aminoglycoside-lipid conjugates is presented.

In patent WO 2012/097454 A1, a class of acetyl transferase (AAC) inhibitors comprised of pantetheine conjugated to the 6′-amine of aminoglycoside has been investigated. In patent US 2014/0357590 A1, newer analogues were synthesized by modifying ring I, ring II, and ring III of pseudotrisaccharide.

Another approach used in patent US 2014/0243280 A1, is use of a novel class of supra molecular protecting groups (SPGs) based on host-guest interaction that offers a new and appealing perspective for single-step modifications based on oligonucleotide.

Another effort done is use of amprolium, as substance secreted by kidneys to prevent toxicity. U.S. Pat. No. 5,691,304 enumerates an improved process for preparing polymyxin B/dextran conjugates where dextran covalently attach to polymyxin B through an amine bond.

Aminoglycosides and polymyxins appear to generate free radicals within the inner ear, with subsequent permanent damage to sensory cells and neurons, resulting in permanent hearing loss. Nephrotoxicity induced by aminoglycosides and polymyxins manifests clinically as non-oliguric renal failure, with a slow rise in serum creatinine and a hypo-osmolar urinary output developing after several days of treatment. Aminoglycosides are nephrotoxic because a small but sizable proportion of the administered dose (≈5%) is retained in the epithelial cells lining the S1 and S2 segments of the proximal tubules after glomerular filtration.

Currently two approaches are used. One of the most simple and radical approaches to reduce polybasic drug nephrotoxicity which would lead to success is decreasing or preventing drug accumulation at organ site. Aminoglycoside/polymyxins accumulation could be reduced either by impairing their uptake or by enhancing their release. Reduction of uptake has been obtained by two strategies.

The first one is aimed at complexing the polybasic drugs extracellularly by conjugation, but conjugation makes drug heavier and result in less excretion and kidney toxicity.

The second one is aimed at competing with or decreasing drug binding to the brush-border membrane but challenge still lies as some polybasic drugs could be their own competitors because uptake by kidney tubular cells is saturable.

In such cases toxicity is reduced by reducing dosing administration frequency of such drugs because drug that passes in the lumen will not be reabsorbed if the drug is too concentrated, which led to conclusion resulting in reducing administration cycle of these polybasic drugs from thrice or twice daily to once daily will the only approach to reduce toxicity.

The problem of toxicity reduction remained unaddressed to a great extent. That is why current inventive work was taken up to address multiple mechanisms and a novel approach of supramolecular cationic complex formation which is without conjugation, without covalent bond formation, without making micelle.

### OBJECTS OF THE INVENTION

One of the main objectives of current invention is to identify compounds which can help reduce multi-organ toxicity and can act by multiple mechanisms.

Another objective is to establish concentration optimization of each such compound in a pre-defined ratio to achieve the target.

Still another objective is to do formulation processes optimization to establish a homeostatic condition such that least or no toxicity is observed in vivo.

Yet another objective is to establish a method of administration of polybasic drugs.

A further objective of current invention is to provide hydrophilic formulations of polybasic drugs without conjugation, without covalent bond formation, without making micelle, to reduce multi-organ toxicity in subject of need which is a mammal.

### SUMMARY OF THE INVENTION

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

One of the aspects of present invention is to provide compositions and formulations for reducing multiorgan toxicities related to polybasic drugs by making supramolecular cationic complex. The compositions and formulations of the present invention have reduced oto-, neuro- and nephro-toxicities related to polybasic drugs.

Another aspect of present invention relates to the processes for the preparation of said supramolecular cationic complex, hereinafter referred to as SMCC compositions and formulations. The SMCC hydrophilic compositions and formulations of the present invention are usefully employed to reduce multiorgan toxicities related to polybasic/cationic drugs in mammals when administered.

In another aspect of present invention, the said supramolecular cationic complex comprise of a polybasic/cationic drug selected from a group of aminoglycoside or polymyxins antibiotics.

In another aspect of present invention, the cationic compound is selected from ethoxylated amines, quaternary ammonium compounds, amino acids l-arginine, l-lysine, histidine.

In another aspect of present invention, a natural polysaccharide is used as scaffold base of supramolecular cationic complex.

In another aspect of the present disclosure, the preparation of the present invention requires polybasic/cationic drug to cationic compound to natural polysaccharide in a ratio of 1:0.1:0.1 to 1:3:1.

In another aspect of the present invention the supramolecular cationic complex of polybasic drugs is formed without any chemical cross linking through electrostatic interactions due to specified charge molecular weight relationship.

Various objects, features, aspects and advantages of the inventive subject matter will become more apparent from the following detailed description of preferred embodiments, along with the accompanying drawing and figures.

### BRIEF DESCRIPTION OF THE DRAWINGS

The embodiments herein will be better understood in terms of their characteristics and effectiveness from the following detailed description with reference to the figures depicting various test results:

FIG. 1: Showing differentiation between normal drug induced toxicity and reduction in SMCC formulations-based toxicities through histopathological studies

FIG. 2 is a graphical representation of Comparative Percentage Change in Kim-1 Values (nephrotoxicity indicating biomarker) from baseline to day 2 after thrice daily administration of control versus various formulations in experimental rat model plasma, according to an embodiment of the present invention.

FIG. 3 is a graphical representation of Comparative Percentage Change in Cystatin-C Values (nephrotoxicity indicating biomarker) from baseline to day 2 after thrice daily administration of control versus various formulations in experimental rat model plasma, according to an embodiment of the present invention.

FIG. 4 is a graphical representation of Comparative Percentage Change in BUN Values (nephrotoxicity indicating biochemical parameter) from baseline to day 2 after thrice daily administration of control versus various formulations in experimental rat model plasma, according to an embodiment of the present invention.

FIG. 5 is a graphical representation of Comparative Percentage Change in Creatinine Values (nephrotoxicity indicating biochemical parameter) from baseline to day 2 after thrice daily administration of control versus various formulations in experimental rat model plasma, according to an embodiment of the present invention.

### DETAILED DESCRIPTION OF THE INVENTION

The embodiments herein and the various features and advantageous details thereof are explained more fully with reference to the non-limiting embodiments that are illustrated in the accompanying figures & tables and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Various terms as used herein are shown below. To the extent a term used in a claim is not defined below, it should be given the broadest definition persons in the pertinent art have given that term as reflected in printed publications and issued patents at the time of filing. Where a definition or use of a term in an incorporated reference is inconsistent or contrary to the definition of that term provided herein, the definition of that term provided herein applies and the definition of that term in the reference does not apply.

It should also be appreciated that the present disclosure can be implemented in numerous ways, including as a composition, a formulation a method of treatment or a method of preparation. In this specification, these implementations, or any other form that the invention may take, may be referred to the compositions and formulations. In general, the compositions or formulations may be altered within the scope of the invention.

For purposes of the present invention the term “Multi-organ toxicity” shall be understood to systemic toxicity in terms of damage to one or more organ such as renal system leading to Acute Kidney Injury (AKI), cell death and kidney failure (Nephrotoxicity), neurotoxicity (Neuromuscular blockade, peripheral neuropathy, paresthesias and encephalopathy induced by polymyxins/aminoglycosides), hearing impairment (Ototoxicity) due to toxicity caused by polybasic drugs.

As used herein the term “Supra Molecular Cationic Complex (SMCC)” refers to a process of making complex, where like charge association through electrostatic interaction is done, whereby cationic compounds/drugs are complexed with like charge molecules by altering their property from basic to acidic or by cation-π interaction and simultaneous physical entrapment of complex such formed on a macromolecular scaffold.

Here it is important to mention that SMCC is not at all correlated with Supramolecular structures, which are large molecules formed by grouping or bonding smaller molecules together and belong to the realm of nanoscience since it is often possible to develop molecules of a desired shape or functionality.

As used herein the term “Macromolecule” refers to a large, organic molecule such as polysaccharides, lipids, proteins, and nucleic acids which are made up of monomer units.

As used herein the term “polybasic/cationic” refers to antibiotic cationic drug compounds which have two or more atoms of replaceable hydrogen for example non ribosomal cyclic lipopeptide antibiotics polymyxins such as but not limited to polymyxins A to E, including polymixin b and colistin and drugs which contain as a portion of the molecule an amino-modified glycoside (sugar) called aminoglycosides such as but not limited to gentamicin, tobramycin, amikacin, plazomicin, streptomycin, neomycin, paromomycin. ribostamycin, arbekacin, dibekacin, gentamycin, kanamycin A and B, aprotinin, aristolochic acid, trichosanthin, etimicin, netilmicin, sisomicin and apramycin.

As used herein the term “adverse drug reaction (ADR)” is any untoward medical occurrence associated with the use of a drug in humans which could be an injury, toxicity, tissue or cell damage, harmful reaction caused by taking medication. ADRs may occur following a single dose or prolonged administration of a drug and is related to organ specific toxicity and could also be multi-organ. Here ototoxicity refers to toxicity in ear cells and associated symptoms like hearing impairment, neurotoxicity refers to neuronal cell damage and related symptoms, nephrotoxicity refers to kidney and renal system cell damage and renal system impairement.

The current invention relates to systemic delivery of SMCC formulations of polybasic drugs to reduce multi-organ toxicity in mammals after administration. Systemic use of several polybasic antibiotic drugs like aminoglycosides, polymyxins A to E is associated with risk of nephrotoxicity, neurotoxicity and ototoxicity. Signs of toxicity appear immediately after administration of high dose or within 24 hr of drug administration irrespective of dosing schedule. Frequent dosing causes more toxicity and more severe ADRs appear, especially in critically ill cases like of sepsis or ICU patients. A lot of studies have been conducted to find ways to reduce toxicity but to a limited success, and the polybasic antibiotic drug products currently being used (herein after referred as reference products) are reported to have signs of toxicity even today, indicating either the methods adopted earlier were in sufficient or had some other challenges which could not reach market. It is now established fact that a major reason for incomplete success to solution for toxicity reduction is presence of multiple mechanisms and no singular approach can cater to reduction in toxicity. Therefore, current invention is designed to cater to an orchestra of cellular mechanisms instead of focusing on a single mechanism being responsible for its etiology.

The current invention disclosure is related to multi organ toxicity reduction including nephrotoxicity reduction of polybasic antibiotic drugs known to cause Acute Kidney Injury (AKI). More particularly, the invention relates to compositions and formulations made thereof, for safer management of oto, neuro and nephro toxicity in critically ill patients or mammals.

According to an embodiment, one of the critical challenges in multiorgan toxicity reduction is prevention of drug accumulation inside organs for example for nephrotoxicity reduction prevention of drug from accumulation inside kidney cells is to be attained.

One of the main causes of polymyxins and aminoglycosides nephrotoxicity is elucidated to their binding with megalin and cubilin receptors present on apical bush border membrane of proximal tubule of kidneys or the cochlea of the inner ear. Polybasic drugs, as defined above, have a high binding affinity for megalin, responsible for drug accumulation leading to ischemia and cell death. These drugs which are basic in nature, they have known affinity for renal/ear/neuronal deposition. The drug when passes through kidney during process of excretion binds to these receptors and gets internalized into epithelial cells and get deposited there.

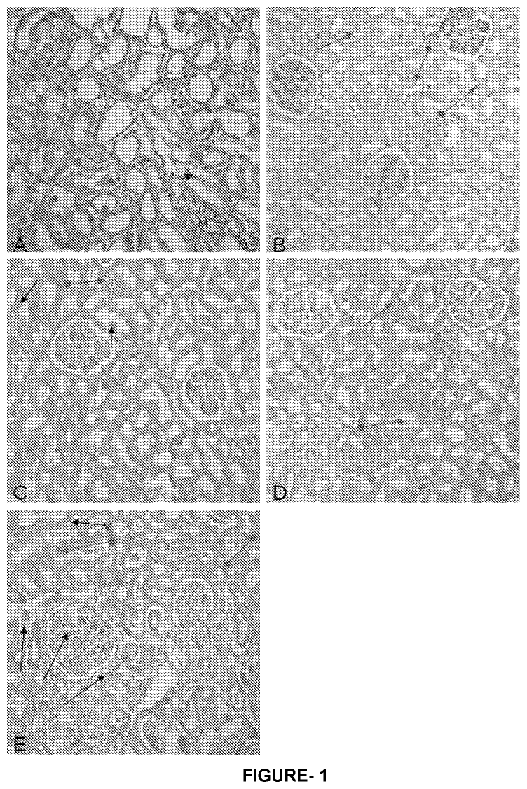

Megalin, an endocytic receptor in the renal proximal tubules, represents the major pathway for accumulation of aminoglycosides/polymyxins in kidney and has been implicated in the development of nephrotoxic acute kidney injury (AKI) by mediating the tubular uptake of nephrotoxic agents. Accumulation of aminoglycosides and Polymyxins in lysosomes with subsequent rupture of the vesicles is considered the main mechanism causing nephrotoxicity in animals and humans. Tubular alterations are associated with the development of focal necroses and apoptosis in the tubular epithelium, together with an extensive tubular and peritubular cell proliferation without an apparent change in kidney function. These toxic signs can be measured by measuring blood biochemical parameters like BUN and creatinine, biomarkers like KIM-1 and Custatin-C and by doing histopathology of related body part. FIG. 1 clearly highlights the histopathological studies showing very less damage (near to normal) condition of renal tubular cells with formulations of current invention.

FIG. 1 (A): shows Acute tubular necroses which are the patchy or diffuse denudation of the renal tubular cells with loss of brush border. Flattening of renal tubular cells due to tubular dilation, intratubular cast formation, vacuolization (arrow end with circle), Congestion (arrow end with line), eosinophilic cytoplasm is seen (arrow end with square). Interstitial mononuclear cellular infiltration (IM), small pyknotic nuclei (arrowhead) in control polymyxin treated rat kidney showing major damage.

FIG. 1 (B): Showing normal glomerular and tubular histology. Mild changes in tubular epithelial lining (arrow end with line), small pyknotic nuclei (arrow end with circle). Tubules show regenerative changes (arrow end with square) in F30 formulation treated rat kidney showing negligible toxicity.

FIG. 1(C): Showing normal glomerular histology. Mild tubular epithelial cell loss, mild congestion (arrow with circle). Tubules show regenerative changes (arrow with line) in F108 formulation treated rat kidney indicating very less toxicity.

FIG. 1 (D): Showing normal glomerular histology. Mild tubular epithelial cell loss, irregular dilated lumina (arrow end with circle). Tubules show regenerative changes (arrow end with line) in F57 treated rat kidney indicating negligible toxicity.

FIG. 1 (E): Showing enlargement of glomerular and acute tubular necrosis with loss of brush border. Flattening of renal tubular cells due to tubular dilation (arrow end with circle), intra tubular cast formation (arrow end with square), vacuolization in tubules (arrow end with V), Congestion (arrow end with line) in control amikacin treated rat kidney showing damaged organ. All three drug groups treated with new formulations of current invention (B:polymyxin F30, C:colistin F108 and D:amikacin F57) exhibited least toxicity even at TID dosing when compared to their respective control (reference) formulations.

One of the embodiments of current invention is to identify compounds which can give competitive binding and restrict polybasic drugs accumulation at organ site.

Another challenge is management of neurotoxicity which causes abnormal neuro-behavioral changes including sensory and motor dysfunction. Neurotoxicity is one major unwanted side-effect (ADRs) associated with polymyxin therapy which is caused due to Polymyxin-induced nerve damage and is largely related to oxidative stress and mitochondrial dysfunction. The central nervous system is very susceptible to oxidative damage due to its obligatory elevated oxygen need. Mitochondria are vital for maintaining basic cellular functions such as energy metabolism, ATP production. Therefore, current invention meticulously managed to alleviate mitochondrial dysfunction by SMCC administration in subjects of need.

Apoptosis plays a critical role in maintaining brain homeostasis in response to drug-induced toxicity. Autophagy involves cellular proteins and organelles engulfing by autophagosomes, digested in lysosomes, and recycled in order to sustain cellular homeostasis in the face of various stresses including nutrient deprivation, hypoxia, oxidative stress, and DNA damage. Autophagy is known to be involved in the maintenance of neuronal homeostasis, particularly in response to drug induced-oxidative stress and mitochondrial dysfunction. The two families of enzymes, NOXs and NOSs are major sources of reactive oxygen species (ROS)/reactive nitrogen species (RNS)/nitrosative stress due to overproduction of nitric oxide (.NO) and act together to damage cells when homeostasis is disturbed. Therefore, reduction of oxidative stress is another important factor managed by current invention by formulating SMCC which maintain homeostasis. Use of appropriate macromolecule which can regulate NO production was another key feature of current invention.

Catalase, Super Oxide Dismutase and Ferric Reducing Ability of Plasma are known oxidative stress markers studied in rat model. A shift towards negative value in control groups indicate rise in oxidative stress. The compositions of current invention and formulations made thereof when tested showed significant rise in antioxidant levels of three parameters tested as represented in examples.

Yet another challenge is management of relative cellular hypoxia, as the major activator of hypoxia-inducible factor, which is detectable in neuro toxicity and chronic kidney disease tissues irrespective of etiology and is thought to result from a combination of structural and functional changes that include; decreased peritubular blood flow associated with glomerular injury, capillary rarefaction, vasoconstriction, luminal narrowing of atherosclerotic vessels, increased oxygen demand from hyperfiltration and tubular hypertrophy, limited oxygen diffusion as a consequence of extracellular matrix expansion, and renal anemia.

Hypoxia is accompanied by a significant increase in blood lactate and severe systemic acidosis as a direct effect of anaerobic metabolism. Therefore, management of hypoxia effectively with regular oxygen supply is also innovatively managed by current technology of SMCC formation and administration in subjects of need.

Another challenge for current invention is to identify a right balance of compounds which when formulated with nephrotoxic polybasic/cationic drugs, can prevent injury or cell damage to a great extent and maintain homeostasis.

According to a preferred embodiment of current invention, for successful competitive inhibition, cationic compounds are chosen to pair with basic drugs like polymyxins and aminoglycosides. Here the cationic compound is selected from group comprising ethoxylated amines, quaternary ammonium compounds, amino acids l-arginine, l-lysine, histidine. According to a preferred embodiment of current invention, the said cationic compound is an amino acid.

Another challenge overcome innovatively in current invention is complexing of like charged molecules as both drug compounds and competitive inhibiting compounds are cationic in nature. This challenge was overcome by experimentally altering the pH of solution to make cationic amino acid to anionic due to zwitterionic nature. Amino acids may be positive, negative, neutral, or polar in nature. At a pH below their pI, they carry a net positive charge; above their pI they carry a net negative charge. So, by altering pH, nature of zwitterionic amino acids can be changed.

Alternatively, the complex formation is done using Cation-π interaction. Several experiments were conducted to find best pair which stabilize formulation while maintaining homoeostasis in vivo to reduce AKI, neurotoxicity and ototoxicity. Of the various Quality By Design (QBD) trials conducted, l-arginine as cationic amino acid was selected for complex formation.

According to another embodiment L arginine is preferred over lysine and histidine. Formulations without L-lysine either failed to stabilize or proved toxic. Additionally, it is important to note that Pka₃ and pI value of L arginine was the highest among the three amino acids and arginine could provide the best competitive inhibition due to highest charge.

| Amino acid | pKa₁ | pKa₂ | pKa₃ | pl |
|---|---|---|---|---|
| Lysine | 2.18 | 8.95 | 10.53 | 9.74 |
| Arginine | 2.17 | 9.04 | 12.48 | 10.76 |
| Histidine | 1.82 | 9.17 | 6.00 | 7.59 |

According to yet another preferred embodiment, both l-arginine and l-lysine offer competitive binding with megalin, but l-arginine is preferred. Arginine is also responsible for NO production and which needs to be regulated, therefore in order to achieve competitive binding while optimizing NO secretion was another challenge managed by current invention skillfully. Concentration optimization of amino acid is critical, and a slight variation disturbs homeostasis required for perfect balancing to reduce toxicity.

According to a preferred embodiment the ratio of said arginine to said drug is between 0.1:1 to 3:1.

According to a preferred embodiment for making supramolecular cationic complex, macromolecule is selected from a group of natural polysaccharide as scaffolding base. The reason for choosing polysaccharide is because they provide continuous source of energy. The selection of polysaccharide was based on charge neutrality. Therefore, selection of a natural polysaccharide played a vital role in decision making.

According to another embodiment, natural polysaccharide is chosen from group comprising Dextran, polysialic acid, pullulans, dextrin, hyaluronic acid, chitosan, and heparins. Other natural polysaccharide agent which can alternatively be used are guar gum, gum arabic, gum tragacanth, larch gum, gum karaya, locust bean gum, agar, alginates, carageenan, pectins, starch, c-starch, xanthan gum, succinoglucan, acrylic acid graft copolymer and the like.

According to yet another preferred embodiment of current invention the macromolecule chosen for SMCC formation is charge neutral plain low molecular weight dextran, which have a linear backbone of α-linked d-glucopyranosyl repeating units. Low molecular weight dextran refers in particular to dextran having <60 KDa as molecular weight. In prior arts dextrans chosen are of high molecular weight ranging from 10⁷ and 10⁸ Kda with high degree of polydispersity, suitable for conjugation and to increase circulation time. These high molecular weight dextrans and their derivatives have been used for drug administration by formation of covalent bond in most prior arts. Rather than going with pre-defined approach, in current invention low molecular weight dextran is chosen without any chemical modification which offers neutral scaffold surface for entrapment of cationic complex.

A main determinant of adequate organ function is the adequate supply and utilization of oxygen at the microcirculatory and cellular level to perform organ function. The highly complex architecture of the renal microvasculature, the need to meet a high energy demand and the fact that the kidney is borderline ischemic, in AKI makes the kidney a highly vulnerable organ to hypoxemic injury. Under normal, steady-state conditions, oxygen (O2) supply to the renal tissues is well regulated; however, under disease or septic conditions the delicate balance of oxygen supply versus demand is disturbed due to renal microvasculature dysfunction.

This dysfunction is largely due to the interaction of renal oxygen handling, nitric oxide metabolism and radical formation. The oxygen requirement of the kidney is mainly determined by the ATP production.

Hence, selection of polysaccharide as macromolecular scaffold was opted as a continuous source of energy & for entrapment of polybasic/cationic drug entity and cationic amino acid to form supramolecular cationic complex, formed by either altering pH of basic amino acid due to zwitterionic nature or by cation-π interaction, such that the complex formed can be entrapped on scaffold of neutral polysaccharide without any chemical bonding, without any conjugation, covalent bond or micelle formulation.

Here it is important to mention that SMCC formation is based on like charge interaction either by altering pH of Arginine due to its zwitterionic nature or by doing cation-π interaction, therefore, ratio of arginine with respect to cationic drug played an important role. Arginine is to competitively bind against drug with receptors, hence ratio of arginine with respect to polymixins (which are more toxic and have higher Pka values than aminoglycosides) is higher than ratio of arginine required against aminoglycosides. The ratio of L arginine to polymixin drugs is between 3:1 to 0.5:1, whereas the ratio of L arginine to aminoglycosides drugs of is between 0.1:1 to 1:1.

Yet another critical and crucial challenge managed for the first time while addressing multiorgan toxicity reduction of polybasic drugs is microcirculatory dysfunction. This can severely limit the ability of the circulation to provide adequate oxygen for fueling oxidative phosphorylation for the production of ATP and can directly impair the function of the Na/K ATPase pump.

However, inflammation and oxidative stress can also severely alter the delicate balance between the oxygen supply and consumption in the brain and kidney. In addition, a disturbance in the homeostasis between reactive oxygen species (ROS), nitric oxide (NO) fueled by neuronal and renal inflammation may contribute to neuro and nephro toxicities.

Several drugs with known neuro and nephrotoxicity are being used in medical practice today due to rising anti-microbial resistance, when no other drug renders result. Continuous supply of energy for ATP production along with microcirculation management is creatively managed by selection of unmodified low molecular weight dextran as polysaccharide as scaffold in SMCC.

According to a preferred embodiment of current invention, a novel feature of current invention is selection of low molecular weight plain Dextran, specifically dextran 40 kDa (herein after referred to as D40 or Dextran 40) is used that too in non-conjugated form without covalent bond formation, without micelle formation, without chemical modification in neutral form for physical entrapment of polybasic drug entity and cationic amino acid to form Supra Molecular Cationic Complex which when administered to subject in need reduces multiorgan toxicity by managing multiple mechanisms responsible to cause toxicity simultaneously.

It is important to mention here that a major disadvantage of conjugation is that it prolongs drug half-life which further increase toxicity. Dextran 40 additionally provides excellent stability with cryoprotection at temperature −45° C. to 60° C. It has been observed that D40 helps in improving blood flow and micro circulation besides it serves as source of energy required for ATP production.

It has been proven through experiments that other forms of low molecular weight Dextran are highly toxic and D40 was found to be the safest neutral polysaccharide of choice. Using D40 in specific ratio which is 100% of drug component, its harmful effects such as accumulation in cells, toxicity is avoided. It is important to highlight that plain dextran is used in current invention without chemical modification, without conjugation or without covalent bond formation.

According to another embodiment, Dextran 40 improved microcirculatory flow by two mechanisms, by decreasing the viscosity of blood by hemodilution and by inhibiting erythrocytic aggregation. Dextran 40 also works as an inhibitor of T lymphocyte adhesion to endothelial cells (EC). Dextran 40 inhibits the constitutive and cytokine-induced binding of T cells to EC, by selectively interfering with the clustering of adhesion molecules on the T cells. This process is thought to play a crucial role in the induction of leucocyte extravasation to the periphery during inflammation.

According to yet another embodiment, addition of one or more sugar-based compound is optional and is at discretion of a person skilled in the art to improve crystalline structure, stability of the formulation depending upon type of processing opted for final formulation.

Besides nephrotoxicity, polybasic drugs are also associated with oto-toxicity. It has been firmly established that polybasic dugs like gentamicin, amikacin, apramycin, plazomycin, polymyxin B, E accumulates in the epithelial cells of cochlea of the inner ear via megalin. Once inside the cell, the drug accumulates in the lysosomes and endoplasmic reticulum, where it binds to calreticulin, leading to elevated levels of misfolded protein in the cell.

After further accumulation, polybasic drug is released into the cytosol, leading to oxidative stress and apoptosis. Therefore, in order to reduce multi-organ toxicity, SMCC complex formed exhibits antioxidant properties to reduce ROS and avoid apoptosis. It has been experimentally proven that D40 forms supra molecular cationic complex (SMCC) with amino acid and polybasic drug to exhibit these properties.

According to one of the most preferred embodiments, the Supra Molecular Cationic Complex is formed due to physical interactions involving electrostatic interactions, without any conjugation, covalent bond or micelle formulation. (FIG. 1-5) clearly differentiate between normal drug induced toxicity and reduction in SMCC formulations-based toxicity.

Resuscitation of the failing kidney needs to integratively correct the homeostasis between oxygen, and reactive oxygen and nitrogen species. Several experimental therapeutic modalities have been performed to prove effectiveness of selective polysaccharides, particularly dextran in restoring microcirculatory oxygenation in parallel to improving oxidative stress and energy source for ATP requirement for Na/K ATP pump functioning to guard renal function following septic AKI.

Dextran 40 is the only dextran that presented with immunomodulatory features and decreased the release of nitric oxide (˜40%) by the cells, both in the absence and presence of lipopolysaccharides (LPS). In addition, Dextran 40 is the more potent than other dextrans in inhibiting lipid peroxidation (70%). These points toward dextran with a 40 kDa weight as being ideal for antioxidant and immunomodulatory use in current invention.

Despite the plethora of beneficial effects discovered to date, warranting a full understanding of the relative benefits and potential adverse effects of L-arginine on human/animal metabolism posed another challenge in current invention.

Some of the key adverse effects of L-arginine include development and/or accelerated growth of certain malignancies. Hence, safe dose optimization to achieve homoeostasis is one of the key inventive steps of current invention. This included by not limited to an essential component of current invention which is required to regulate NO levels produced by 1-arginine. Arginine plays a vital role in competitive inhibition with megalin.

Therefore, selection of specific weight and molar ratios which proved less toxic in combination with other components is carefully studied by a series of experiments. Innovatively for the first-time arginine, despite being like charged with polybasic drugs, is rendered charge modification by pH alteration so that a complex can be formed. Hence, concentration optimization becomes an integral part of inventive step of current invention.

Yet another challenge overcome is key serious adverse effects of dextran which included among others anaphylaxis, volume overload, pulmonary edema, cerebral edema, or platelet dysfunction, hypotension, shock and cardiac arrest. An uncommon but significant complication of dextran osmotic effect is acute renal failure.

Therefore, another important inventive step of current invention is selection of right molecular weight of dextran and then concentration optimization of dextran which can be used to achieve its antioxidant effect with improved micro circulation, controlling NO production besides providing source of energy required to prevent AKI and multi-organ toxicity.

According to a preferred embodiment of current invention the said ratio of arginine to D40 is between 0.25:1 to 7.5:1.

According to yet another embodiment, the ratio of said cationic complex to macromolecule is 1:0.05 to 1:0.5 in SMCC.

According to yet another important embodiment, maintain a charge molecular weight balance is essential for SMCC to achieve homeostasis inside body and to provide stability of compositions and formulations made thereof till atleast 24 months (end of shelf life).

Therefore, based on the Flory-Huggins theory for entropy term and the Debye-Hückel theory for electrical/electrostatic interaction term, when σ³r≥0.53 (σ is charge density/charge per unit area and r is polymer molecular weight) the condition for complexation to occur is fulfilled which in current invention is ≥53, proving complex is feasible.

According to yet another one of the most important embodiments of current invention is the ratio of each component in the said complex. Ratio is very critical to achieve homeostasis after administration to the subject in need. After a series of experiments to check toxicity reduction in animals, and after verifying the best formulation to achieve maximum antioxidant effect the ratios of each component in SMCC were optimized.

According to a preferred embodiment, the ratio of said cationic drug to said cationic amino acid to low molecular weight dextran is 1:0.1:0.1 to 1:3:1 in SMCC formed.

According to yet another preferred embodiment, the ratio of said cationic drug to said cationic amino acid to low molecular weight dextran is 1:0.1:0.2 to 1:3:0.75 in SMCC formed.

According to one of the most preferred embodiments of current invention, the said polybasic/cationic drug used for making SMCC composition and formulation made thereof are polymyxin B and polymyxin E or a pharmaceutical salt thereof which are electrostatically combined with cationic amino acid L arginine using cation-π interaction while entrapping the said cationic complex formed in low molecular weight dextran D40.

According to yet another one of the most preferred embodiments of current invention, the said polybasic/cationic drug used for making SMCC composition and formulation made thereof are amikacin and aparmycin or a pharmaceutical salt thereof which are electrostatically combined with cationic amino acid L arginine by pH alteration of arginine and simulatenous entrapping the said cationic complex formed in low molecular weight dextran D40.

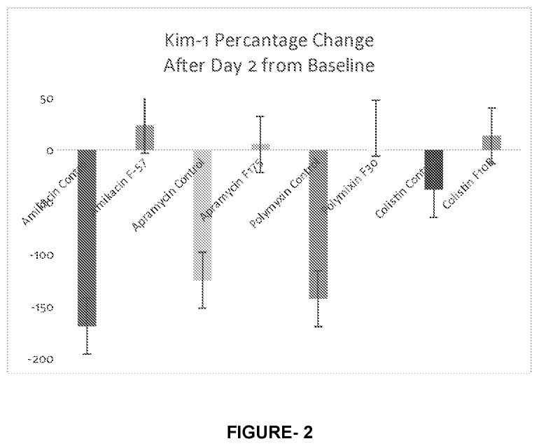

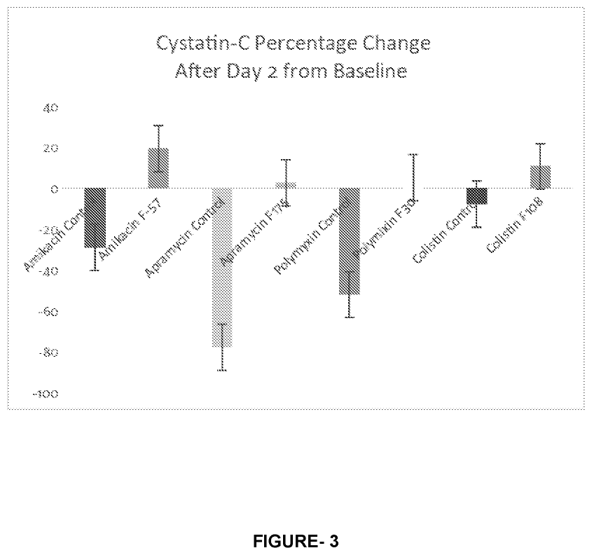

According to yet another most preferred embodiment of current invention, for early recognition of acute kidney injury is done through use of biomarkers such as Kidney injury molecule 1 (KIM-1) and Cystatin C. KIM-1 is highly upregulated in proximal tubular cells following kidney injury. The serum level of cystatin C is a stronger predictor of the renal outcome and the risk of cardiovascular toxicity than the creatinine level. In FIGS. 2 and 3 clearly indicate that when Amikacin 400 mg/kg*3 times daily of each Control and F57, Apramycin 500 mg/kg*3 times daily each of control and F175, Polymyxin 7.5 mg/kg*3 times Control and F30, Colistin 12 mg/kg*3 times daily each of Control and F108 were administered, all control groups damaged kidney and a negative trend in graph is observed for Kim-1 and cystatin-C, whereas formulations of current inventions showed positive bar trend, indicating least kidney damage.

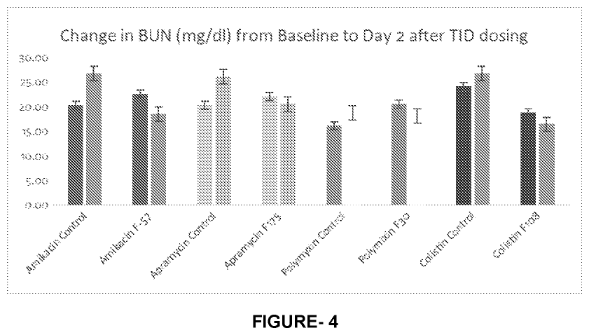

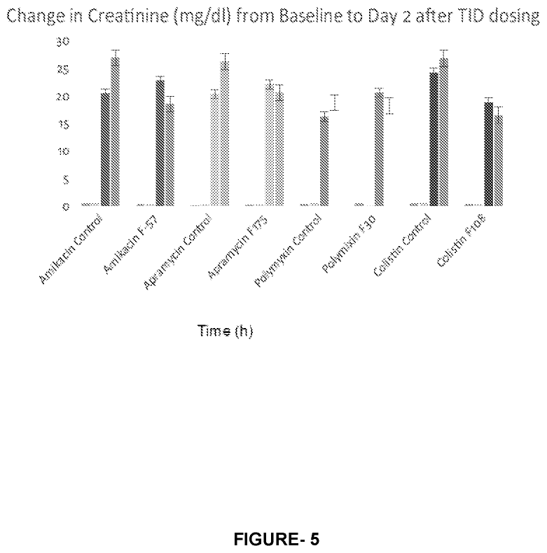

According to one the most preferred embodiment of current invention, various formulations of polybasic drugs have been optimized for maintenance of homeostasis after administration to reduce multiorgan toxicity. FIGS. 4 and 5 represent comparison with reference drugs. In an experiment different rat groups were administered several test formulations in respective TID dosing along with reference control drugs (marketed reference product) for 2 days. Pre and post dosing samples were collected from each group. Biochemical parameters BUN and creatinine were measured for Kidney function test in plasma/serum. The formulation F-30 of Polymixin B, F-108 of colistin, F-57 of amikacin and F-175 of apramycin found to have less/no effects on serum biochemical levels which was found elevated significantly in marketed formulation at same dose levels, indicating there is significantly high kidney damage in control groups of all polybasic drugs.

According to yet another embodiment of current invention, addition of any of Lysine/histidine with arginine increased toxicity. A slight deviation in charge neutralization potential results in higher toxicity in comparison to the best ratio optimized by current invention. Inventor from a series of experiments concluded that toxicity reduction is significantly lesser in any other combination or ratio or replacing arginine with other cationic amino acids than optimized in current invention.

According to another preferred embodiment the ratio of said drug to said macromolecule is 1:0.1 to 1:1 in SMCC. It is important to mention here that removal of any of the component from the said complex results in higher toxicity especially removal of D40 or reduction beyond one tenth of drug concentration results in toxicity even higher than original drug probably because concentration below that fails to maintain nitric oxide balance, provide sufficient antioxidant effect and the resultant drug product toxicity is further enhanced by high arginine caused toxicity. An experiment was conducted to test Nitrotyrosine levels in blood of rats comparing control versus selected formulations of current invention. An increase in rise of Nitrotyrosine values is observed in control group indicative of nitrosative stress which is significantly reduced with current invention compositions and formulations made thereof.

According to yet another preferred embodiment of current invention, a concentration of D40 more than equal to drug product does not make complex feasible during formulation and complex such formed are not stable. Replacement of D40 with dextran 20 KDa or dextran 60 KDa resulted in very high mortality. Repeated experiments have proven that among all D40 was safest for tox reduction.

According to yet another one of the most important embodiments of current invention is the said supramolecular complex is formed by altering arginine pH due to its zwitterionic nature to enable cationic complex formation.

According to another important embodiments of current invention is that alternatively the said supramolecular complex is formed by cation-7 interaction.

According to yet another embodiment of current invention the ratio of the said cationic amino acid arginine to said macromolecule varies from the nature of binding. In polymyxin drug case of cationic pie interaction, the said ratio of arginine to D40 in polymyxin polybasic drugs compositions and formulations made thereof is between 7.5:1 to 2.5:1.

According to yet another preferred embodiment in aminoglycosides where arginine charge is altered by altering pH due to its zwitterionic nature before bonding to drug, in such cases the ratio of arginine to D40 is between 0.25:1 to 1:1. Increasing arginine beyond dextran amount in such formulations result in drastic increase in nitrosative and oxidative stress, resulting in higher toxicity.

According to another embodiment the said complex is administered to a subject in need by parenteral route and the said subject is preferably a mammal.

According to a preferred embodiment the said polybasic/cationic drug used for making SMCC composition and formulation made thereof is polymyxin B or a pharmaceutical salt thereof which is electrostatically combined with cationic amino acid L arginine along with low molecular weight dextran D40 in the ratio of said drug:cationic amino acid:dextran is 1:1.4:0.2 to 1:2.5:0.5.

According to another preferred embodiment the said polybasic/cationic drug used for making SMCC composition and formulation made thereof is polymyxin E or a pharmaceutical salt thereof, which is electrostatically combined with cationic amino acid L arginine along with low molecular weight dextran D40 providing scaffold for physical entrapment and the ratio of said drug:cationic amino acid:dextran is 1:0.5:0.1 to 1:2:0.25.

According to yet another preferred embodiment the said polybasic/cationic drug used for making SMCC composition and formulation made thereof is amikacin or a pharmaceutical salt thereof which is electrostatically combined with L arginine along with D40 in the ratio of said drug:cationic amino acid:dextran is 1:0.2:0.3 to 1:0.5:0.75.

According to another preferred embodiment the said polybasic/cationic drug used for making SMCC composition and formulation made thereof is apramycin or a pharmaceutical salt thereof which is combined with L arginine along with D40 in the ratio of said drug:cationic amino acid:dextran is 1:0.1:0.2 to 1:0.5:0.75.

According to another important embodiments of the current invention, where said supramolecular cationic complex maintains homeostasis when administered parenterally to mammals to reduce multi organ toxicity without disturbing kinetics of individual drug. The said compositions are formulated as liquid or lyophilized formulation.

The following examples are given to illustrate the invention. It should be understood, however, that the invention is not to be limited to the specific conditions or details described in these examples:

### Example 1—SMCC Formulations Optimization Studies

Formulation examples depicting ratios

| | | | | | Cationic amino | | | |
|---|---|---|---|---|---|---|---|---|
| Formulation | Drug name | | | | acid | | Polysaccharide | |
| no | PolyB | Colistin | Amikacin | Apramycin | Arginine | Lysine | D20 | D40 |
| F14 | 1 | — | — | — | — | 1.01 | — | 0.03 |
| F15 | 1 | — | — | — | 1.09 | — | — | — |
| F16 | 1 | — | — | — | 1.2 | 1.18 | — | — |
| F29 | 1 | — | — | — | — | — | — | 0.16 |
| F28 | 1 | — | — | — | — | 1.2 | — | — |
| F25 | 1 | — | — | — | 1.26 | — | — | 0.18 |
| F26 | 1 | — | — | — | 1.4 | — | — | 0.2 |
| F30 | 1 | — | — | — | 1.48 | — | — | 0.27 |
| F33 | 1 | — | — | — | 2.5 | — | — | 0.5 |
| F51 | — | — | 1 | — | 0.12 | 1.12 | — | — |
| F52 | — | — | 1 | — | 0.14 | — | 0.22 | — |
| F53 | — | — | 1 | — | 0.14 | — | — | 0.26 |
| F55 | — | — | 1 | — | | 0.16 | — | 0.29 |
| F56 | — | — | 1 | — | 0.2 | — | — | 0.3 |
| F57 | — | — | 1 | — | 0.25 | — | — | 0.32 |
| F75 | — | — | 1 | — | 0.5 | — | — | 0.75 |
| F78 | — | — | 1 | — | 0.1 | — | — | 1 |
| F101 | — | 1 | — | — | 0.15 | 0.09 | — | 0.04 |
| F103 | — | 1 | — | — | 0.21 | — | — | 0.05 |
| F104 | — | 1 | — | — | 0.26 | 0.12 | — | 0.05 |
| F105 | — | 1 | — | — | 0.41 | — | — | — |
| F107 | — | 1 | — | — | 0.5 | — | — | 0.1 |
| F108 | — | 1 | — | — | 0.66 | — | — | 0.15 |
| F111 | — | 1 | — | — | 3 | — | — | 0.5 |
| F152 | — | — | — | 1 | 0.01 | — | — | - |
| F155 | — | — | — | 1 | — | 0.11 | — | 0.09 |
| F160 | — | — | — | 1 | 0.05 | — | — | 0.13 |
| F171 | — | — | — | 1 | 0.06 | 1.2 | 0.01 | — |
| F172 | — | — | — | 1 | 0.09 | — | — | 0.19 |
| F174 | — | — | — | 1 | 0.1 | — | — | 0.2 |
| F175 | — | — | — | 1 | 0.22 | — | — | 0.29 |
| F187 | — | — | — | 1 | 0.5 | — | — | 0.75 |
| F189 | — | — | — | 1 | 0.85 | — | — | 1 |

### Example 2—Comparative Plasma Oxidative Stress Markers Studies in Rat Model with Reference Drugs and Formulations of Current Inventions

Comparative Antioxidants Potential

| | Catalase Activity (IU/mg of Protein) | | | SOD Activity (IU/mg of Protein) | | | FRAP (mMol of FE++) | | | Nitrotyrosine (ng/ml) | | |
|---|---|---|---|---|---|---|---|---|---|---|---|---|
| | Average values | | | Average values | | | Average values | | | Average values | | |
| | | Day | % | | Day | % | | Day | % | | Day | % |
| Drug groups | Baseline | 2 | change | Baseline | 2 | change | Baseline | 2 | change | Baseline | 2 | change |
| Amikacin | 0.61 | 0.55 | −9.6 | 2.96 | 2.45 | −17.4 | 36.52 | 29.78 | −18.46 | 12.53 | 28.84 | 130.17 |
| (400 | | | | | | | | | | | | |
| mg/kg*3times) | | | | | | | | | | | | |
| Control | | | | | | | | | | | | |
| Amikacin | 0.58 | 1.04 | 80.18 | 2.57 | 2.59 | 0.49 | 34.12 | 39.14 | 14.71 | 12.42 | 12.34 | −0.64 |
| (400 | | | | | | | | | | | | |
| mg/kg*3times) | | | | | | | | | | | | |
| F-57 | | | | | | | | | | | | |
| Amikacin | 0.72 | 0.67 | −6.94 | 2.65 | 1.36 | −48.68 | 34.42 | 15.86 | −53.92 | 12.84 | 29.85 | 132.48 |
| (500 mg/kg * | | | | | | | | | | | | |
| 3 times) | | | | | | | | | | | | |
| Control | | | | | | | | | | | | |
| Amikacin | 0.65 | 0.89 | 36.92 | 2.49 | 3.56 | 42.97 | 35.14 | 38.85 | 10.56 | 13.01 | 13.12 | 0.85 |
| (500 mg/kg * | | | | | | | | | | | | |
| 3 times) | | | | | | | | | | | | |
| F175 | | | | | | | | | | | | |
| Polymyxin | 0.8 | 0.54 | −32.02 | 2.85 | 2.04 | −28.54 | 34.18 | 10.76 | −68.52 | 13.12 | 32.43 | 147.18 |
| 7.5 mg/kg*3 | | | | | | | | | | | | |
| times) | | | | | | | | | | | | |
| Control | | | | | | | | | | | | |
| Polymyxin | 0.6 | 0.89 | 33.14 | 2.37 | 3.41 | 44.16 | 35.44 | 41.94 | 18.34 | 12.91 | 11.5 | −10.92 |
| 7.5 mg/kg | | | | | | | | | | | | |
| *3 times), | | | | | | | | | | | | |
| F30 | | | | | | | | | | | | |
| Colistin | 0.59 | 0.22 | −62.49 | 2.69 | 2.27 | −15.59 | 33.88 | 22.45 | −33.74 | 13.31 | 347.3 | 180.24 |
| 12 mg/kg *3 | | | | | | | | | | | | |
| times | | | | | | | | | | | | |
| Control | | | | | | | | | | | | |
| Colistin | 0.49 | 0.79 | 58.89 | 2.49 | 3.47 | 44.49 | 32.56 | 38.63 | 18.64 | 13.45 | 12.2 | −9.29 |
| 12 mg/kg *3 | | | | | | | | | | | | |
| times, F108 | | | | | | | | | | | | |

According to above study Antioxidant potentials of each formulation is evaluated in serum of rats used for the nephrotoxicity evaluation study. Changes in antioxidant parameters like Catalase activity, SOD (Superoxide dismutase) activity and Ferrous ion reducing antioxidant power (FRAP) in serum were evaluated using well established procedure and compared with reference drug groups. After TID dosing of each drug (reference drug/marketed formulation) the results of % change is observed in negative which is indicative of rising oxidative stress. Formulation groups of current invention of each drug tested showed a significantly decreased in oxidative stress represented by positive percentage change, indicating antioxidant capacity of drug, thereby reducing toxicity. Test for Nitrotyrosine indicates that nitrosative stress increased with administration of all drug control groups and is reduced drastically to <1 or negative values with formulations of current invention indicating the role of homeostasis maintained by components of current compositions.

### Example 3: Comparative Antioxidant Study of Control Drug Groups Versus Formulations with and without Arginine and Dextran

| | | Total Equivalent | Superoxide | | |
|---|---|---|---|---|---|
| | | Antioxidant | radical | Scavanging of | Reducing |
| | | Capacity | scavenging | hydrogen | Power |
| Name of | | (TEAC) assay | assay | Peroxide | Assay |
| formulation | Type | % inhibition in oxidative stress | | | |
| Polymixin | Control | 29.34 | 26.32 | 25.34 | 19.21 |
| F30 | Final | 67.38 | 71.25 | 68.54 | 59.54 |
| | Without | 40.54 | 38.54 | 32.34 | 27.54 |
| | Arginine | | | | |
| | Without | 44.54 | 46.52 | 55.84 | 49.21 |
| | Dextran | | | | |
| Colistin | Control | 26.28 | 28.65 | 24.6 | 20.31 |
| F108 | Final | 69.67 | 70.38 | 66.54 | 55.32 |
| | Without | 38.64 | 36.29 | 30.94 | 25.64 |
| | Arginine | | | | |
| | Without | 42.34 | 44.68 | 51.85 | 46.21 |
| | Dextran | | | | |
| Amikacin | Control | 25.54 | 23.52 | 22.85 | 18.59 |
| F 57 | Final | 65.21 | 66.54 | 67.54 | 62.54 |
| | Without | 43.86 | 40.84 | 48.27 | 45.21 |
| | Arginine | | | | |
| | Without | 37.01 | 34.54 | 36.84 | 23.54 |
| | Dextran | | | | |
| Apramycin | Control | 27.84 | 27.38 | 26.76 | 17.54 |
| F 175 | Final | 66.64 | 70.64 | 64.21 | 58.29 |
| | Without | 40.95 | 41.54 | 51.64 | 45.31 |
| | Arginine | | | | |
| | Without | 34.85 | 35.95 | 29.64 | 27.54 |
| | Dextran | | | | |

As per above study an in vitro experiment was conducted to test Antioxidant potentials of each formulations of current invention and with and without arginine and dextran and were compared with reference products. Test parameters included Total Equivalent Antioxidant Capacity (TEAC) assay, super oxide anion radical scavenging, hydrogen peroxide radical scavenging and reducing power assays. Results demonstrated that F-30, F-108, F-57 and F-175 had significantly higher scavenging activity when compared with control. When formulations without arginine were compared, the antioxidant potential was still higher than control in aminoglycosides. When formulations without dextran were compared, the antioxidant potential was significantly higher than control in Polymixins groups, indicating that final formulations having specified ratio of each excipient has the highest antioxidant potential. This experiment clearly demonstrated that without Arginine or without Dextran the free radicals rise significantly indicating a major role of each ingredient in maintaining homeostasis.

### KEY FEATURES OF THE INVENTION

The present invention provides novel compositions and formulations of polybasic drugs to reduce multi-organ toxicities.

The present invention provides supramolecular cationic complex compositions and formulations of like charged molecules without any chemical crosslinking or covalent bond formation.

The present invention provides concentration optimization of each compound in a pre-defined ratio to achieve the best possible effects and homeostasis after drug administration which provides least or reduced toxicity.

The present invention provides compositions for supramolecular cationic complex formation with very selective choices of cationic compound and macromolecule to target multiple mechanisms simultaneously so that a near perfect balancing is achieved in vivo.

I claim:

1. Compositions and formulations made of polybasic drugs for reducing multi organ toxicities in mammals, by forming supramolecular cationic complex without chemical cross linking, wherein such complexes comprise of:
(a) a polybasic/cationic drug selected from a group of aminoglycoside or polymyxins antibiotics;
(b) a cationic compound selected from group of ethoxylated amines, quaternary ammonium compounds, amino acids
wherein the said amino acid is selected from 1-arginine, 1-lysine, histidine;
(c) a macromolecule for scaffold base;
wherein the said macromolecule is selected from a group of natural polysaccharide such as dextran, polysialic acid, pullulans, dextrin, hyaluronic acid, chitosan, and heparin,
wherein the said macromolecule is a low molecular weight dextran without chemical modification;
wherein the said complex is formed by cationic electrostatic interactions in specified charge molecular weight relationship;
wherein the ratio of said cationic drug:cationic amino acid:low molecular weight dextran is 1:0.1:0.1 to 1:3:1;
(d) wherein the said complex is administered to a subject in need by parenteral route.

2. Composition of claim 1, wherein the said cationic amino acid is L arginine.

3. Composition of claim 1, wherein the said low molecular weight dextran is dextran 40 KDa.

4. Compositions of claim 1 and formulations made thereof,
wherein the said supra molecular cationic complex is formed due to physical connections involving electrostatic interactions, without any conjugation, covalent bond or micelle formulation;
wherein the ratio of said drug to said macromolecule is 1:0.1 to 1:1;
wherein the ratio of said arginine to said drug is between 0.1:1 to 3:1;
wherein the said supramolecular cationic complex maintains homeostasis when administered parenterally to mammals to reduce multi organ toxicity.

5. Composition of claim 1, wherein the said cationic drug is polymyxin B or a pharmaceutical salt thereof which is electrostatically combined with cationic amino acid L arginine along with low molecular weight dextran D40 providing scaffold for physical entrapment;
wherein the ratio of said drug:cationic amino acid:dextran is 1:1.4:0.2 to 1:2.5:0.5.

6. Composition of claim 1, wherein the said cationic drug is polymyxin E or a pharmaceutical salt thereof which is electrostatically combined with cationic amino acid L arginine along with low molecular weight dextran D40 providing scaffold for physical entrapment;
wherein the ratio of said drug:cationic amino acid:dextran is 1:0.5:0.1 to 1:2:0.25.

7. Composition of claim 1, wherein the said cationic drug is amikacin or a pharmaceutical salt thereof which is electrostatically combined with cationic amino acid L arginine along with low molecular weight dextran D40 providing scaffold for physical entrapment;
wherein the ratio of said drug:cationic amino acid:dextran is 1:0.2:0.3 to 1:0.5:0.75.

8. Composition of claim 1, wherein the said cationic drug is apramycin or a pharmaceutical salt thereof which is electrostatically combined with cationic amino acid L arginine along with low molecular weight dextran D40 providing scaffold for physical entrapment;
wherein the ratio of said drug:cationic amino acid:dextran is 1:0.1:0.2 to 1:0.5:0.75.

9. Composition of claim 1, wherein the said supramolecular complex is formed by altering pH of arginine with simultaneous complex formation;
wherein the ratio of said cationic amino acid to said macromolecule in supramolecular cationic complex formation of aminoglycoside polybasic drugs is between 0.25:1 to 1:1.

10. Composition of claim 1, wherein the said supramolecular complex is formed by cation-π interaction;
wherein the ratio of cationic amino acid to macromolecule in supramolecular cationic complex formation of polymyxin polybasic drugs is between 7.5:1 to 2.5:1.
